(19) United States
(12) Patent Application Publication
(10) Pub. No.: US 2025/0215516 A1
(43) Pub. Date: Jul. 3, 2025

(54) COLORIMETRIC LOOP-MEDIATED ISOTHERMAL AMPLIFICATION SYSTEM

(71) Applicant: Delta Electronics Int’l (Singapore) Pte Ltd, Singapore (SG)

(72) Inventors: Zee Hong Goh, Singapore (SG); Jia Hui Esther Boey, Singapore (SG); Weishi Zhang, Singapore (SG)

(21) Appl. No.: 18/400,352

(22) Filed: Dec. 29, 2023

(51) Int. Cl. C12Q 1/70; C12Q 1/6844; G01N 21/78

(52) U.S. Cl. CPC C12Q 1/701; C12Q 1/6844; G01N 21/78

(57) ABSTRACT

A colorimetric LAMP system includes a colorimetric LAMP reaction mixture and an extraction-free lysis buffer. The colorimetric LAMP reaction mixture is all-in-one lyophilized and includes a primer set, a strand-displacing polymerase and deoxyribonucleoside triphosphates for amplifying a target sequence; a pH indicating dye in a concentration ranged 0.08 to 0.3 mM; and a lyoprotectant sugar in a concentration ranged 1 to 10% (w/v), wherein the lyoprotectant sugar is selected from the group consisting of trehalose, raffinose, dextran, mannitol and mixtures thereof. The extraction-free lysis buffer includes potassium chloride in a concentration ranged 10 to 50 mM; ammonium sulfate in a concentration ranged 10 to 50 mM; and a detergent in a concentration ranged 0.5 to 6% (w/v). The lyophilized colorimetric LAMP reaction mixture is rehydrated with the extraction-free lysis buffer to be ready for nucleic acid amplification and detection.

### FIELD OF THE INVENTION

The present disclosure relates to a loop-mediated isothermal amplification (LAMP) system, and more particularly to a colorimetric LAMP system.

### BACKGROUND OF THE INVENTION

Disease diagnosis can be commonly classified as disease antigen detection, disease nucleic acid detection or even antibody detection. Using COVID-19 as an example, there are currently two primary types of tests being used: molecular test (also known as nucleic acid test (NAT)) and antigen rapid test (ART). The NAT which employs qPCR or qRT-PCR technologies serves as a gold standard in some countries as it is more accurate than the ART. However, the trade-off is that most traditional NAT results come back in around 24 hours, and some may take longer depending on if they're sent to an external laboratory. While for the ART, the results can be obtained in as little as 15 minutes, and it is much cheaper to produce the ART kit. The problem, though, is that the ART is not as sensitive as the NAT (˜1000 times less sensitive), so it is more likely to receive false negative and false positive results suggesting low specificity, sensitivity and reliability of the ART when comparing with the NAT.

Many types of the NAT have been developed but there exist some drawbacks. For example, long turnaround time in the traditional NAT reduces the effectiveness in early diagnostic and protection measurement in pathogen transmission. Additionally, the traditional NAT is bulky, costly and sophisticated and thus is unfavorable for lay person usage and home-based setting. Another type of the NAT with short turnaround time and lower capital investment known as loop-mediated isothermal amplification (LAMP) was developed in 2000. The designs of the LAMP allow the user to detect the amplification status via turbidimeter or fluorescence detector. Both detection methods require significant capital investment which increases the cost to be used as a home-based device. A further type of the NAT uses colorimetric detection of nucleic acid amplification whereby the reaction mixture includes enzyme and halochromic agent which allow the user to differentiate the sample status via naked eye due to the change of color. However, the reagent recipe contains high concentration of anti-freeze component such as betaine and glycerol which prevent the reagent to be freeze-dried, suggesting the transportation of the reagent must be in cold chain to prevent enzyme degradation. This reduces the chance of being used as a home-based device due to the reagent stability. Although freeze-dried compositions have been developed, the PCR components are lyophilized separately for enzyme and primer parts. This increases the process procedure and prolongs the manufacturing duration.

Apart from the reagent stability which requires lyophilization to achieve home-based setting, another arising issue of the NAT will be the sample purity. The major drawback for the NAT is the sample input for the test requires high purity which involves complete nucleic extraction and purification. Although various methods have been developed for high quality nucleic acid extraction, it poses risk to the sample as the process during extraction might “nick” the nucleic acids and increase the chance of affecting the sensitivity of LAMP due to longer amplicon size. Furthermore, the user needs to invest in procuring the correct extraction kit before using the assay. This further prolongs the diagnostic time and limits the diagnostic to be only conducted in the laboratory with sophisticated machinery set up.

Therefore, there is a need of providing a nucleic acid detection system to address the challenges encountered in the prior arts.

### SUMMARY OF THE INVENTION

An object of the present disclosure is to provide a colorimetric LAMP system for rapid, sensitive and specific diagnosis of viral genomic materials via naked eyes.

Another object of the present disclosure is to provide a colorimetric LAMP reaction mixture which allows the user to lyophilize the whole mixture to achieve temperature stability.

An additional object of the present disclosure is to provide an extraction-free lysis buffer which is compatible to the colorimetric LAMP reaction mixture.

In accordance with an aspect of the present disclosure, a colorimetric LAMP system is provided. The colorimetric LAMP system includes a colorimetric LAMP reaction mixture and an extraction-free lysis buffer. The colorimetric LAMP reaction mixture is all-in-one lyophilized and includes a primer set, a strand-displacing polymerase and deoxyribonucleoside triphosphates for amplifying a target sequence; a pH indicating dye in a concentration ranged 0.08 to 0.3 mM; and a lyoprotectant sugar in a concentration ranged 1 to 10% (w/v), wherein the lyoprotectant sugar is selected from the group consisting of trehalose, raffinose, dextran, mannitol and mixtures thereof. The extraction-free lysis buffer includes potassium chloride in a concentration ranged 10 to 50 mM; ammonium sulfate in a concentration ranged 10 to 50 mM; and a detergent in a concentration ranged 0.5 to 6% (w/v), wherein the detergent is selected from the group consisting of 2-ethylhexan-1-ol;2-methyloxirane;oxirane (CAS number: 64366-70-7), secondary alcohol ethoxylate (CAS number: 84133-50-6), 2-[4-(2,4,4-trimethylpentan-2-yl)phenoxy]ethanol (CAS number: 9002-93-1), 2-[4-(2,4,4-trimethylpentan-2-yl)phenoxy]ethan-1-ol (CAS number: 9002-93-1), and {2-[3,4-bis(2-hydroxyethoxy)oxolan-2-yl]-2-(2-hydroxyethoxy)ethoxy}ethyl dodecanoate (CAS number: 9005-64-5) and mixtures thereof. The lyophilized colorimetric LAMP reaction mixture is rehydrated with the extraction-free lysis buffer to be ready for nucleic acid amplification and detection.

In an embodiment, the colorimetric LAMP reaction mixture further includes a reverse transcriptase for converting a viral genomic RNA to a complementary DNA.

In an embodiment, the pH indicating dye is phenol red or neutral red.

In an embodiment, a volume of the colorimetric LAMP reaction mixture is less than 10 μl.

In an embodiment, the colorimetric LAMP reaction mixture includes 2 to 7.5% (w/v) of trehalose and 2 to 7.5% (w/v) of raffinose.

In an embodiment, the colorimetric LAMP reaction mixture includes 2 to 7.5% (w/v) of trehalose, 2 to 7.5% (w/v) of raffinose, 1 to 2.5% (w/v) of dextran, and 1 to 5% (w/v) of mannitol.

In an embodiment, the colorimetric LAMP reaction mixture further includes a magnesium salt in a concentration ranged 4 to 12 mM.

In an embodiment, the detergent in the extraction-free lysis buffer is 0.5 to 6% (w/v) of 2-ethylhexan-1-ol;2-methyloxirane;oxirane (CAS number: 64366-70-7).

In an embodiment, the detergent in the extraction-free lysis buffer is 0.5 to 6% (w/v) of secondary alcohol ethoxylate (CAS number: 84133-50-6).

In an embodiment, the detergent in the extraction-free lysis buffer is 0.5 to 6% (w/v) of 2-[4-(2,4,4-trimethylpentan-2-yl)phenoxy]ethanol (CAS number: 9002-93-1).

In an embodiment, the detergent in the extraction-free lysis buffer is 0.5 to 6% (w/v) of 2-[4-(2,4,4-trimethylpentan-2-yl)phenoxy]ethan-1-ol (CAS number: 9002-93-1).

In an embodiment, the detergent in the extraction-free lysis buffer is 0.5 to 6% (w/v) of {2-[3,4-bis(2-hydroxyethoxy)oxolan-2-yl]-2-(2-hydroxyethoxy)ethoxy}ethyl dodecanoate (CAS number: 9005-64-5).

The above contents of the present disclosure will become more readily apparent to those ordinarily skilled in the art after reviewing the following detailed description and accompanying drawings, in which:

### BRIEF DESCRIPTION OF THE DRAWINGS

FIG. 1A shows the color change performance of the colorimetric LAMP using the phenol red as the pH indicating dye;

FIG. 1B shows the color change performance of the colorimetric LAMP using the neutral red as the pH indicating dye;

FIG. 2A shows the lyo-bead in the microcentrifuge tube;

FIG. 2B shows the lyo-cake in the 8-well strip tube;

FIG. 3 shows the rehydration of the lyo-bead and the color change after amplification;

FIG. 4 shows the performance comparison between the fresh colorimetric LAMP reaction mixture and the lyo-bead colorimetric LAMP reaction mixture;

FIG. 5 shows the performance comparison between the commercial RT-LAMP and the lyo-cake colorimetric LAMP;

FIG. 6 shows the performance comparison between the lyo-bead and the lyo-cake using the same set of samples;

FIG. 7A shows the real-time stability test at room temperature for the colorimetric LAMP reaction mixture;

FIG. 7B shows the endpoint RFU;

FIG. 7C shows the Cq values of the stability test;

FIG. 8A to FIG. 8E show performance of various detergents in lysing the live virus HCoV-OC43;

FIG. 9A to FIG. 9C show the performance of the direct lysis buffer on different viruses; and

FIG. 10A and FIG. 10B show real time stability and performance of the direct lysis buffer for the extracted RNA and the live virus, respectively.

### DETAILED DESCRIPTION OF THE PREFERRED EMBODIMENT

The present disclosure will now be described more specifically with reference to the following embodiments. It is to be noted that the following descriptions of the embodiments of this disclosure are presented herein for purpose of illustration and description only; it is not intended to be exhaustive or to be limited to the precise form disclosed.

The present disclosure utilizes a modified loop-mediated isothermal amplification (LAMP) with a pH indicator, also known as colorimetric LAMP, for rapid, sensitive and specific diagnosis of viral genomic materials via naked eyes. There is provided an assay kit and a method which requires LAMP for detecting the viral genomic materials. The method includes a reverse transcription of converting a viral genomic RNA from the virus to a complementary DNA (cDNA) and then an isothermal amplification whereby the cDNA is amplified with a primer set.

Particularly, the present disclosure provides a colorimetric LAMP system. The colorimetric LAMP system includes a colorimetric LAMP reaction mixture which allows the user to lyophilize the whole mixture to achieve temperature stability, and an extraction-free lysis buffer, also called direct lysis buffer, which is compatible to the colorimetric LAMP reaction mixture. The present disclosure relates to a recipe to produce lyophilized colorimetric LAMP reaction mixture in bead or cake form, and a protocol for direct nucleic acid extraction without purification. The recipes of the colorimetric LAMP reaction mixture and the direct lysis buffer are described in detail as follows.

The colorimetric LAMP reaction mixture includes an enzyme and materials for nucleic acid amplification, a pH indicating dye, and a sugar. The enzyme is a strand-displacing polymerase, and the materials for nucleic acid amplification includes a primer set and deoxyribonucleoside triphosphates (dNTPs) for amplifying a target sequence. The pH indicating dye is capable of providing color change in visual light to allow the user to differentiate the amplification results. The sugar is a lyoprotectant sugar with specific concentration and is able to protect the colorimetric LAMP reaction mixture especially the enzyme from degradation during the freeze-drying process. By including the optimal lyoprotectant sugar, the colorimetric LAMP reaction mixture of the present disclosure is able to be all-in-one lyophilized into a lyophilized ready-to-use reagent after the freeze-drying process.

The direct lysis buffer includes special selections of a chaotropic salt and a detergent for ideal extraction of nucleic acids. The direct lysis buffer is capable of lysis and direct nucleic acid extraction of viruses, such as respiratory viruses including influenza virus A (FluA), influenza virus B (FluB), respiratory syncytial virus (RSV), severe acute respiratory syndrome coronavirus 2 (SARS-CoV-2), and human coronavirus OC43 (HCoV-OC43), but not limited thereto. The lyophilized colorimetric LAMP reaction mixture is able to be rehydrated with the direct lysis buffer and ready for nucleic acid amplification and detection.

In an embodiment, the colorimetric LAMP reaction mixture includes the primer set, the strand-displacing polymerase and the deoxyribonucleoside triphosphates (dNTPs) for amplifying the target sequence, the pH indicating dye in a concentration ranged 0.08 to 0.3 mM, and the lyoprotectant sugar in a concentration ranged 1 to 10% (w/v). The lyoprotectant sugar is selected from the group consisting of trehalose, raffinose, dextran, mannitol and mixtures thereof.

In an embodiment, the colorimetric LAMP reaction mixture further includes the reverse transcriptase for converting the viral genomic RNA to complementary DNA.

In an embodiment, the pH indicating dye is phenol red or neutral red, but not limited thereto.

In an embodiment, the volume of the colorimetric LAMP reaction mixture may be ranged 8 to 20 μl. Particularly, the volume of the colorimetric LAMP reaction mixture can be reduced to less than 10 μl, such as 8 to 10 μl, which is beneficial to reduce the volume of the lyophilized bead or cake, compared to the standard of 20 μl.

In an embodiment, the colorimetric LAMP reaction mixture includes 2 to 7.5% (w/v) of trehalose and raffinose, respectively, in a 9.5 μl of the colorimetric LAMP reaction mixture as the lyoprotectant.

In an embodiment, a 60% (w/v) of D-(+)-trehalose dihydrate and 30% (w/v) of D-(+)-raffinose pentahydrate are prepared and dissolved in molecular grade water, and the final concentration of the lyoprotectant in the colorimetric LAMP reaction mixture is 2 to 7.5% (w/v) each.

In an embodiment, the colorimetric LAMP reaction mixture includes 2 to 7.5% (w/v) of trehalose, 2 to 7.5% (w/v) of raffinose, 1 to 2.5% (w/v) of dextran, and 1 to 5% (w/v) of mannitol, in a 9.5 μl of the colorimetric LAMP reaction mixture as the lyoprotectant.

In an embodiment, a magnesium salt, such as magnesium sulfate, in a concentration ranged 4 to 12 mM is further included in the colorimetric LAMP reaction mixture. The magnesium salt facilitates the primer to target and bind tightly to the target.

In an embodiment, an intercalating fluorescence dye, such as EvaGreen® dye, is further added into the colorimetric LAMP reaction mixture for quality control or detection for fluorescence LAMP.

An illustrative recipe of the colorimetric LAMP reaction mixture for SARS-CoV-2 detection is shown in Table 1, and distilled water is further added to a total volume of 9.5 μl.

TABLE 1

| Component | Final concentration | |
|---|---|---|
| dNTPs | 1.0-1.4 | mM |
| Primer | 0.16-3.2 | μM |
| EvaGreen ® | 0.3-1.0 | x |
| Strand-displacing polymerase | 0.2-0.6 | unit/μl |
| Reverse transcriptase | 0.10-0.3 | unit/μl |
| MgSO₄ | 4-12 | mM |
| pH indicating dye (pH 8.8) | 0.08-0.3 | mM |
| Trehalose | 2-7.5% | (w/v) |
| Raffinose | 2-7.5% | (w/v) |
| Dextran | 1-2.5% | (w/v) |
| Mannitol | 1-5% | (w/v) |

Apart from the colorimetric LAMP assay development, the present disclosure also includes the development of colorimetric LAMP-friendly direct lysis buffer. The direct lysis buffer allows direct amplification without further purification after the viruses are lysed and the genomic materials are released into the buffer, so that the total assay time is reduced.

The present disclosure provides a recipe of the direct lysis buffer to release the genomic materials of the viruses, and the lysis buffer harboring the genomic materials can be directly added to the above-mentioned colorimetric LAMP reaction mixture without further purification.

The direct lysis buffer of the present disclosure utilizes an optimized buffer composition including detergents and LAMP tolerable chemicals to achieve virus lysis and amplification.

In an embodiment, the direct lysis buffer of the present disclosure includes potassium chloride (KCl) in a concentration ranged 10 to 50 mM, ammonium sulfate [(NH₄)₂SO₄] in a concentration ranged 10 to 50 mM, and a detergent in a concentration ranged 0.5 to 6% (w/v). The detergent is selected from the group consisting of 2-ethylhexan-1-ol;2-methyloxirane;oxirane (Ecosurf™ EH-9, CAS number: 64366-70-7), secondary alcohol ethoxylate (Tergitol™ Type 15 S-7 or Type 15 S-9, CAS number: 84133-50-6), 2-[4-(2,4,4-trimethylpentan-2-yl)phenoxy]ethanol (Triton® X-100, CAS number: 9002-93-1), 2-[4-(2,4,4-trimethylpentan-2-yl)phenoxy]ethan-1-ol (IGEPAL®, CAS number: 9002-93-1), and {2-[3,4-bis(2-hydroxyethoxy)oxolan-2-yl]-2-(2-hydroxyethoxy)ethoxy}ethyl dodecanoate (Tween® 20, CAS number: 9005-64-5) and mixtures thereof. This composition makes up the direct lysis buffer friendly to the colorimetric LAMP reaction.

An illustrative recipe of the direct lysis buffer used with the lyophilized colorimetric LAMP reaction mixture mentioned above is shown in Table 2, and the pH value of the direct lysis buffer is 8.0 to 9.0 which is adjusted using KOH and HCl.

TABLE 2

| Component | | Final concentration | |
|---|---|---|---|
| Potassium chloride | | 10-50 | mM |
| Ammonium sulfate | | 10-50 | mM |
| Detergent | Ecosurf ™ EH-9 | 0.5-6% | (w/v) |

Tergitol ™ Type 15 S-7
Tergitol ™ Type 15 S-9
Triton ® X-100
IGEPAL ®
Tween ® 20

In an embodiment, the direct lysis buffer includes 10 to 50 mM of potassium chloride, 10 to 50 mM of ammonium sulfate, and 0.5 to 6% (w/v) of Ecosurf™ EH-9. This composition makes up the direct lysis buffer friendly to the colorimetric LAMP reaction with capability to release the genomic materials from SARS-CoV-2 or HCoV-OC43.

In an embodiment, a 100 to 500 mM of potassium chloride and ammonium sulfate master mixture are prepared and diluted with molecular grade water. The pH of the master mixture is adjusted with 10N KOH and 1N HCl to pH 8.1 to 8.8 (Note that “N” is used to represent the equivalent concentration or normality). Then, the direct lysis buffer is prepared using the master mixture pH 8.5 with 10% (w/v) Ecosurf™ EH-9 to form the direct lysis buffer including 10 to 50 mM of potassium chloride, 10 to 50 mM of ammonium sulfate, and 0.5 to 6.% (w/v) of Ecosurf™ EH-9.

In an embodiment, the direct lysis buffer includes 10 to 50 mM of potassium chloride, 10 to 50 mM of ammonium sulfate, and 0.5 to 6% (w/v) of Tergitol™ Type 15 S-7.

In an embodiment, the direct lysis buffer includes 10 to 50 mM of potassium chloride, 10 to 50 mM of ammonium sulfate, and 0.5 to 6% (w/v) of Tergitol™ Type 15 S-9.

In an embodiment, the direct lysis buffer includes 10 to 50 mM of potassium chloride, 10 to 50 mM of ammonium sulfate, and 0.5 to 6% (w/v) of Triton® X-100.

In an embodiment, the direct lysis buffer includes 10 to 50 mM of potassium chloride, 10 to 50 mM of ammonium sulfate, and 0.5 to 6% (w/v) of IGEPAL®.

In an embodiment, the direct lysis buffer includes 10 to 50 mM of potassium chloride, 10 to 50 mM of ammonium sulfate, and 0.5 to 6% (w/v) of Tween® 20.

The following are examples illustrating the applications of the colorimetric LAMP reaction mixture and the direct lysis buffer of the present disclosure.

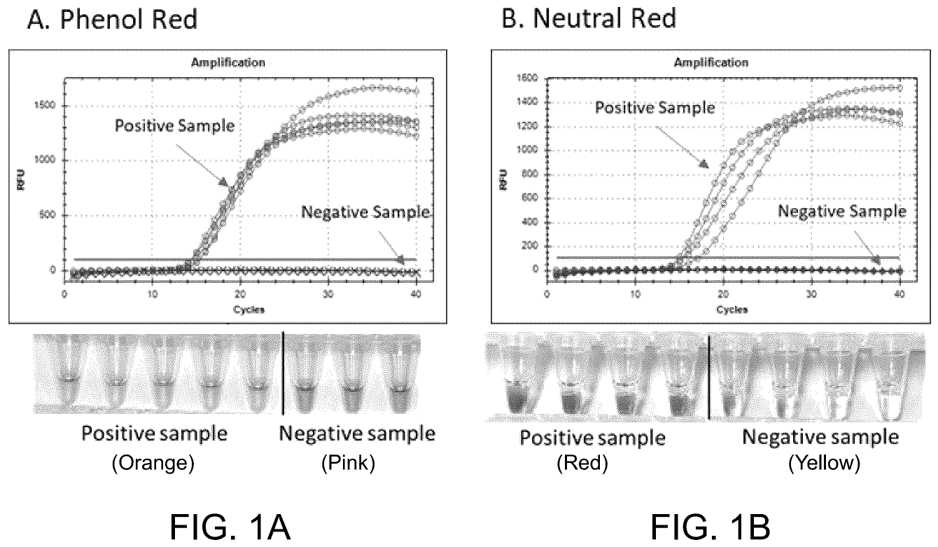

Example 1 illustrates the comparison of the fluorescence LAMP with the colorimetric LAMP using SARS-CoV-2 reference RNA. The colorimetric LAMP reaction mixture mentioned above and the pH indicating dye of phenol red and neutral red were used for the colorimetric LAMP assay. An intercalating fluorescence dye was added into the recipe for the fluorescence LAMP assay. FIG. 1A shows the color change performance of the colorimetric LAMP using the phenol red as the pH indicating dye, while FIG. 1B shows the color change performance of the colorimetric LAMP using the neutral red as the pH indicating dye. The upper parts of FIG. 1A and FIG. 1B show the amplification curves of the fluorescence LAMP assays, while the lower parts of FIG. 1A and FIG. 1B show the visual results of the colorimetric LAMP assay. For the phenol red, the color of the reaction mixture turns orange/yellow for the positive sample or remains pink for the negative sample after amplification. For the neutral red, the color of the reaction mixture turns red for the positive sample or turns yellow for the negative sample after amplification. As shown in FIG. 1A and FIG. 1B, the color changes of the colorimetric LAMP were in concordance to the amplification results of the fluorescence LAMP. The duration of the colorimetric LAMP reaction was 20 minutes which was equivalent to 40 Cq in the amplification cycle.

The colorimetric LAMP reaction mixture of the present disclosure utilizes an optimized buffer composition, resulting in a rapid diagnosis turnaround time of 20 minutes. All these are achieved without compromising on the sensitivity of SARS-CoV-2 kit detection.

Further, the colorimetric LAMP reaction mixture of the present disclosure is lyophilized ready-to-use recipe which allows the colorimetric LAMP reaction mixture to be freeze-dried and stable in room temperature. The recipe is able to be lyophilized inside a strip tube to form a lyo-cake or using a bead dispensing machine to form a lyo-bead without changing the composition.

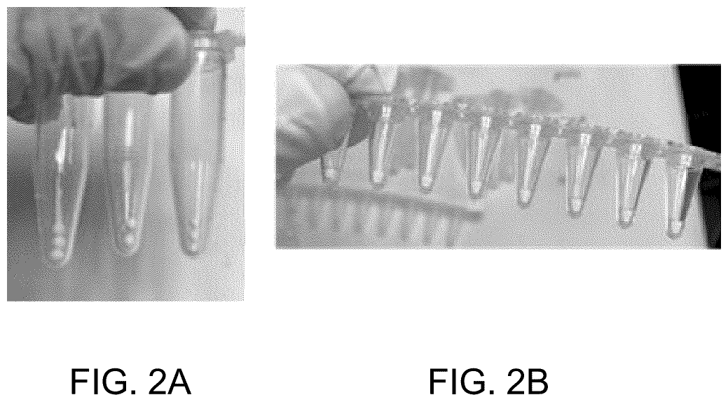

Example 2 illustrates the lyophilized colorimetric LAMP reaction mixture of the present disclosure. FIG. 2A shows the lyo-bead in the microcentrifuge tube and FIG. 2B shows the lyo-cake in the 8-well strip tube. The lyo-bead and the lyo-cake shared the same lyophilization recipe using lyoprotectant concentration listed in the above embodiments.

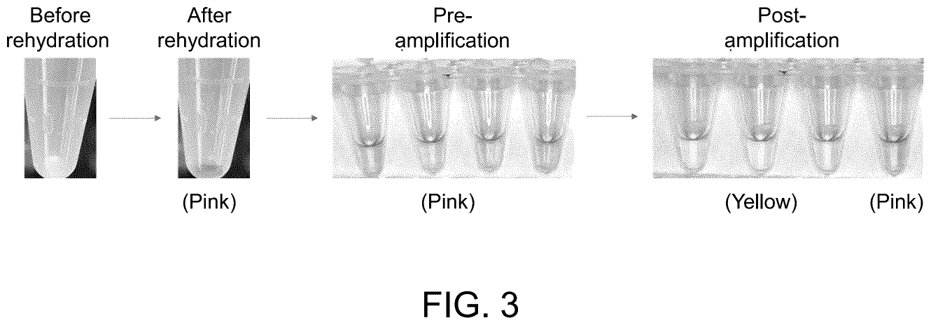

Example 3 illustrates the rehydration of the lyophilized colorimetric LAMP reaction mixture. As mentioned above, the lyophilized colorimetric LAMP reaction mixture can be easily rehydrated by the direct lysis buffer. In this example, 9.5 μl of the colorimetric LAMP reaction mixture was lyophilized into the lyo-bead, and the phenol red was used as the pH indicating dye. FIG. 3 shows the rehydration of the lyo-bead and the color change after amplification. The lyo-bead was first rehydrated with the direct lysis buffer and showed to revert back to its original pink color. After amplification, the color changed to yellow or orange for positive amplification. Further, the lyophilized colorimetric LAMP reaction mixture either lyo-bead or lyo-cake showed similar performance after rehydrated by the direct lysis buffer using fluorescence signal as indicator of performance.

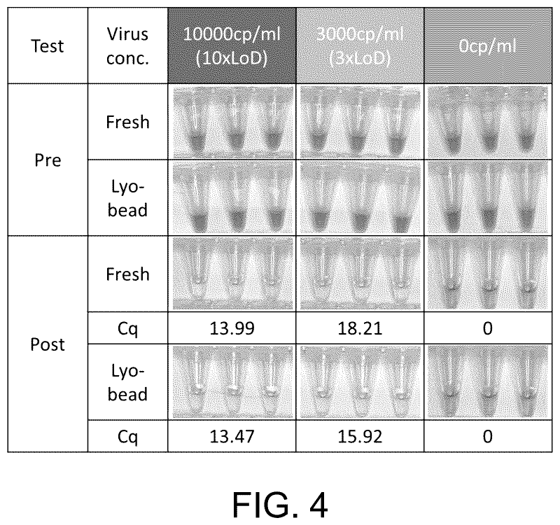

Example 4 illustrates the performance comparison between fresh and lyo-bead colorimetric LAMP reaction mixtures using SARS-CoV-2 reference RNA. The intercalating fluorescence dye was added into the colorimetric LAMP reaction mixture to use fluorescence signal as indicator of performance. In this example, the performance of the lyo-bead colorimetric LAMP reaction mixture was compared with the freshly prepared liquid colorimetric LAMP reaction mixture, and the phenol red was used as the pH indicating dye. The test virus concentrations were 10000 cp/ml (10×LoD, Limit of Detection), 3000 cp/ml (3×LoD), and 0 cp/ml. FIG. 4 shows the performance comparison between the fresh colorimetric LAMP reaction mixture and the lyo-bead colorimetric LAMP reaction mixture. It shows that the color changed to yellow only for the positive samples with Cq values of 13.99, 13.47, 18.21 and 15.92, while others remain pink. Therefore, comparable sensitivity can be observed between the fresh and the lyo-bead reagents. Further, the fluorescence Cq value for the lyo-bead showed a 2Cq earlier comparing with the fresh reagent.

As a result, the recipe of the colorimetric LAMP reaction mixture of the present disclosure is able to lyophilize the colorimetric LAMP reaction mixture without losing the performance for the colorimetric LAMP assay after rehydration when compared with the freshly prepared colorimetric LAMP reaction mixture.

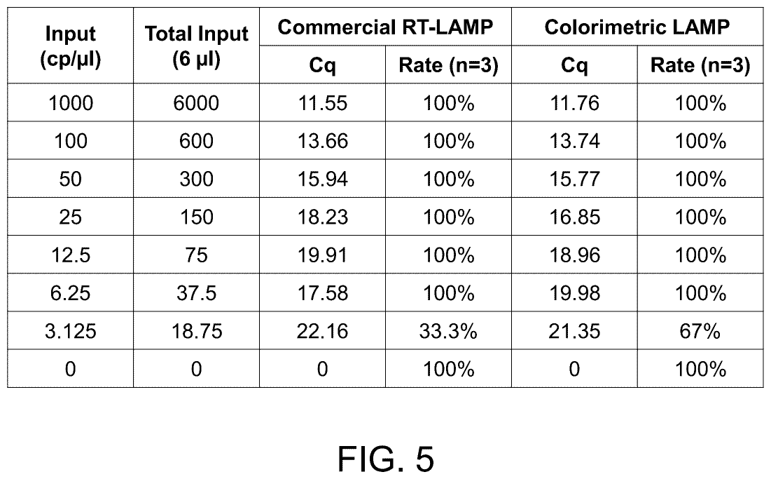

Example 5 illustrates the performance comparison between the present lyophilized colorimetric LAMP reaction mixture and the commercial RT-LAMP reagent using SARS-CoV-2 reference RNA. The intercalating fluorescence dye was added into the colorimetric LAMP reaction mixture to use fluorescence signal as indicator of performance. In this example, the colorimetric LAMP reaction mixture includes 5% (w/v) of trehalose and 5% (w/v) of raffinose as the lyoprotectant. The lyo-cake colorimetric LAMP reaction mixture was rehydrated with the direct lysis buffer. FIG. 5 shows the performance comparison between the commercial RT-LAMP and the lyo-cake colorimetric LAMP. As a result, the lyo-cake colorimetric LAMP reaction mixture coupled with the direct lysis buffer showed comparable performance as the commercial RT-LAMP reagent, and comparable sensitivity with the RT-LAMP reagent with 100% detection rate using 37.5 copies/reaction of the reference RNA.

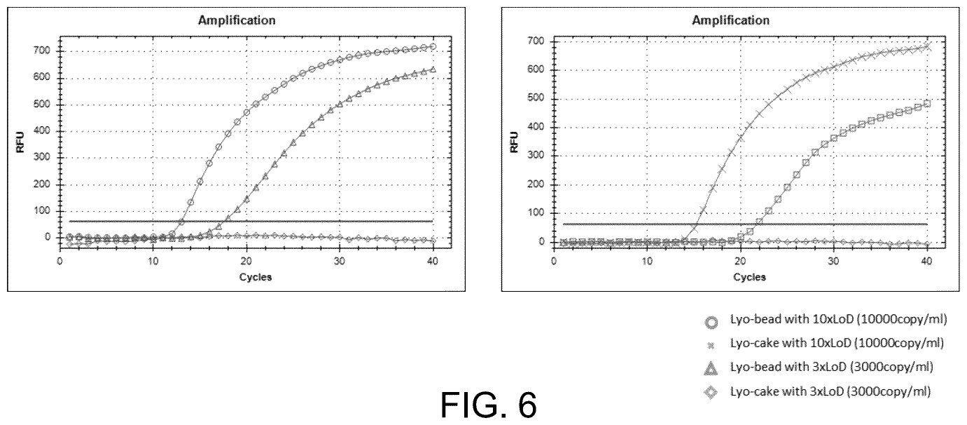

Example 6 illustrates the performance comparison between the lyo-bead and the lyo-cake colorimetric LAMP reaction mixtures. The intercalating fluorescence dye was added into the colorimetric LAMP reaction mixture to use fluorescence signal as indicator of performance. In this example, the lyo-bead and the lyo-cake shared the same lyophilization recipe using lyoprotectant concentration of Example 5. The test virus concentrations were 10000 cp/ml (10×LoD) and 3000 cp/ml (3×LoD). FIG. 6 shows the performance comparison between the lyo-bead and the lyo-cake using the same set of samples. As a result, the lyo-bead and the lyo-cake colorimetric LAMP reaction mixtures show comparable performance.

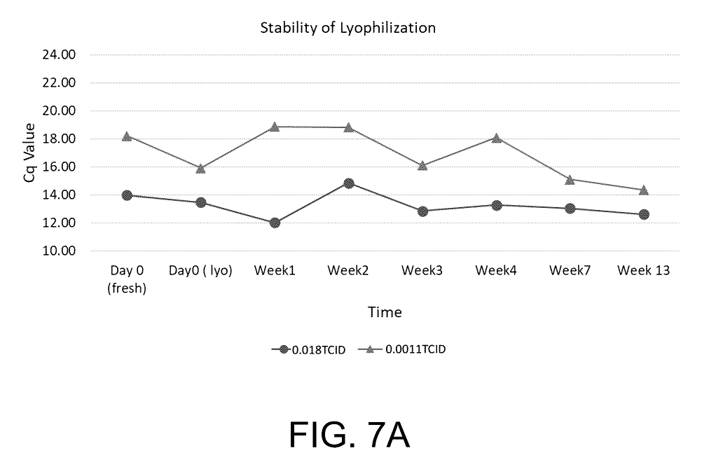

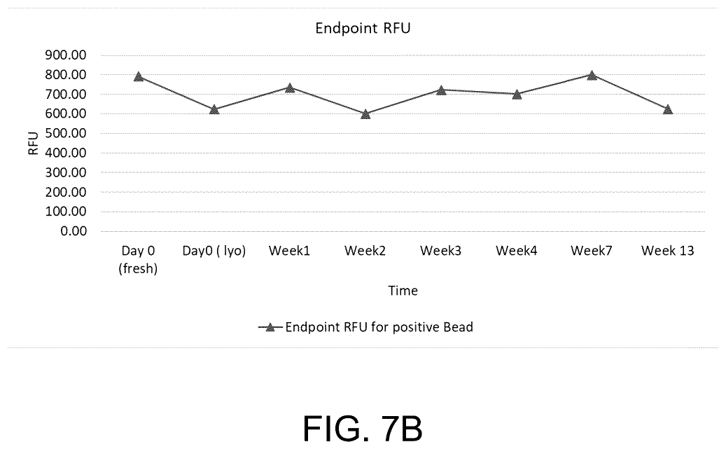

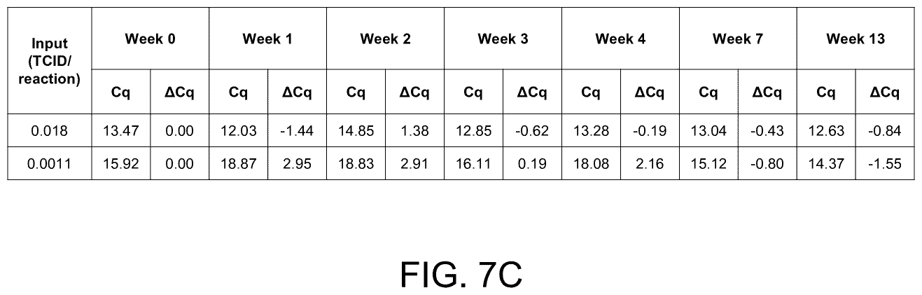

Example 7 illustrates the stability of the lyophilized colorimetric LAMP reaction mixture of the present disclosure. The intercalating fluorescence dye was added into the colorimetric LAMP reaction mixture to use fluorescence signal as indicator of performance. In this example, the recipe was also used for SARS-CoV-2 detection, and both low concentration sample (0.0011TCID50) and high concentration sample (0.018TCID50) were tested. FIG. 7A shows the real-time stability test at room temperature for the colorimetric LAMP reaction mixture, FIG. 7B shows the endpoint RFU (relative fluorescence unit), and FIG. 7C shows the Cq values of the stability test. As a result, the lyo-bead performance remained stable until the 13th week for both low and high concentration samples. The endpoint RFU suggested that the stable signal was obtained until the 13th week. Therefore, the lyophilized colorimetric LAMP reaction mixture of the present disclosure is able to be stored at room temperature at least to the 13th week without losing its performance.

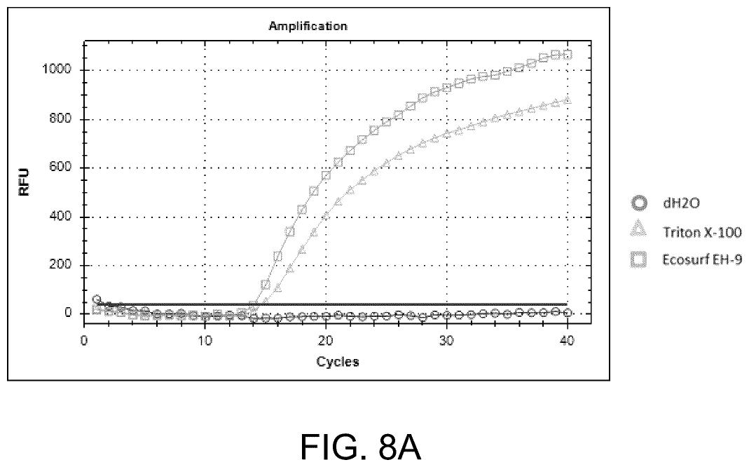

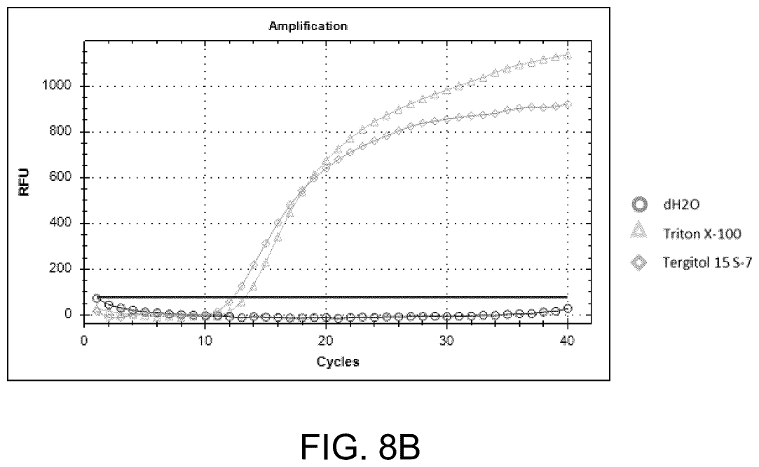

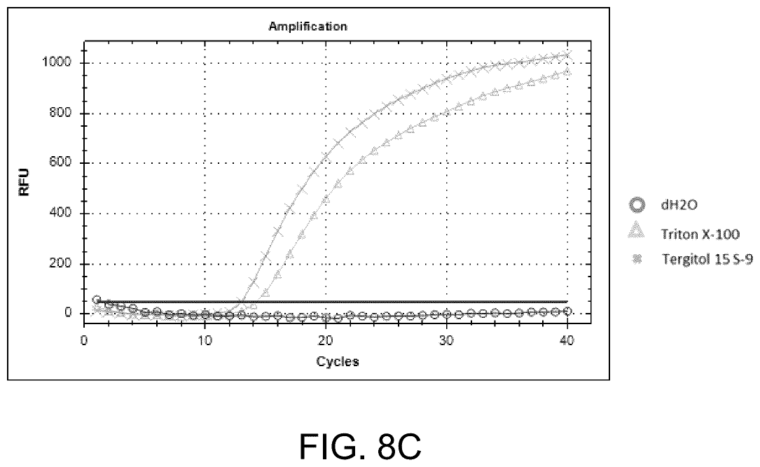

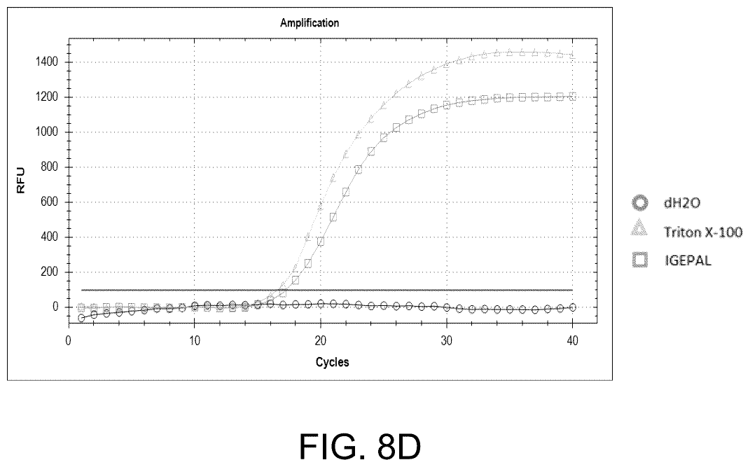

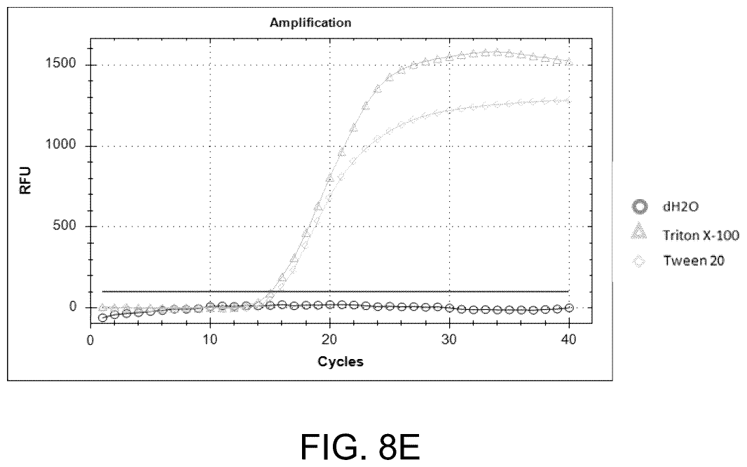

Example 8 illustrates the performance of the direct lysis buffer produced using different detergent and used with the lyophilized colorimetric LAMP reaction mixture. The intercalating fluorescence dye was added into the colorimetric LAMP reaction mixture to use fluorescence signal as indicator of performance. In this example, the direct lysis buffer included one detergent, which was Triton® X-100, Ecosurf™ EH-9, Tergitol™ Type 15 S-7, Tergitol™ Type 15 S-9, IGEPAL®, or Tween® 20, and the optimal concentration range for each detergent was 0.5 to 6% (w/v). The direct lysis buffer was used to lyse the live virus HCoV-OC43, which may represent SARS-CoV-2 due to both are human corona viruses. FIG. 8A to FIG. 8E show performance of various detergents in lysing the live virus HCoV-OC43. From the figures, it is clear that each of the selected detergents is able to release the viral genomic materials and promote the LAMP reaction. Therefore, the direct lysis buffer of the present disclosure has capability to release the genomic materials from viruses in comparison with water (dH₂O) as lysis buffer.

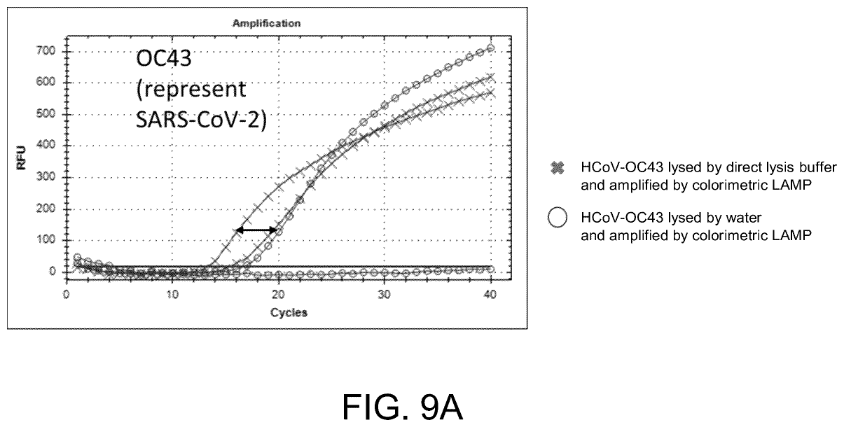

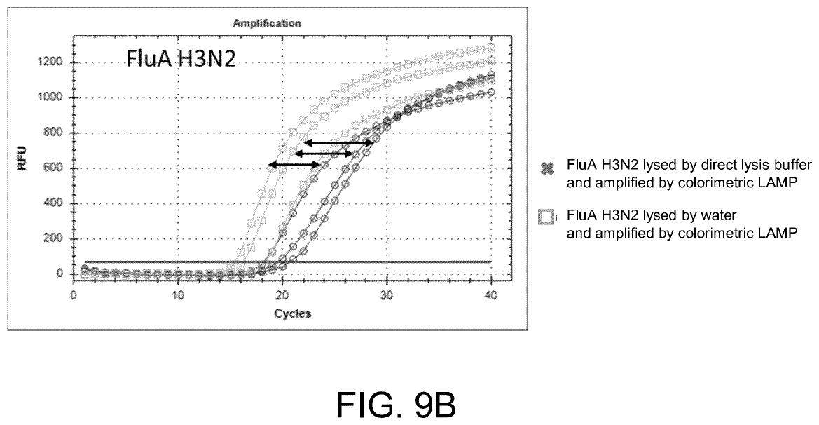

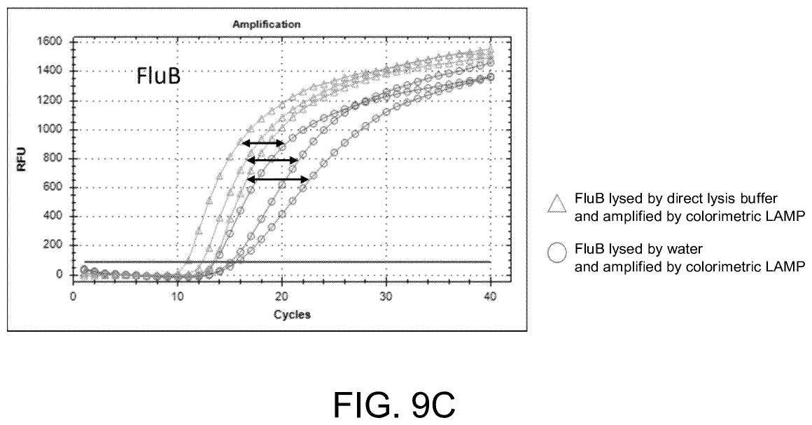

Example 9 illustrates the performance of the direct lysis buffer used with the lyophilized colorimetric LAMP reaction mixture on various live virus strains. The intercalating fluorescence dye was added into the colorimetric LAMP reaction mixture to use fluorescence signal as indicator of performance. In this example, the live viruses of HCoV-OC43, FluA H3N2 and FluB were spiked into the direct lysis buffer, respectively, and later mixed with the colorimetric LAMP reaction mixture for amplification. FIG. 9A to FIG. 9C show the performance of the direct lysis buffer on different viruses. As shown in the figures, earlier Cq can be detected for the viruses lysed by the direct lysis buffer, suggesting the direct lysis buffer is able to lyse the live viruses and release more genomic materials for amplification, and thus improve the sensitivity of the colorimetric LAMP assay compared with the viruses lysed by water (dH₂O). Additionally, the lowest input concentration was able to be 100% detected on the sample for HCoV-OC43 lysed by the direct lysis buffer, while only 50% detection was observed on the sample for HCoV-OC43 lysed by water.

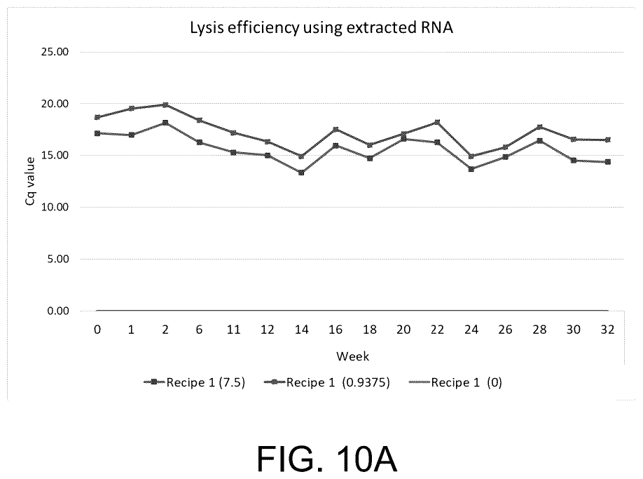

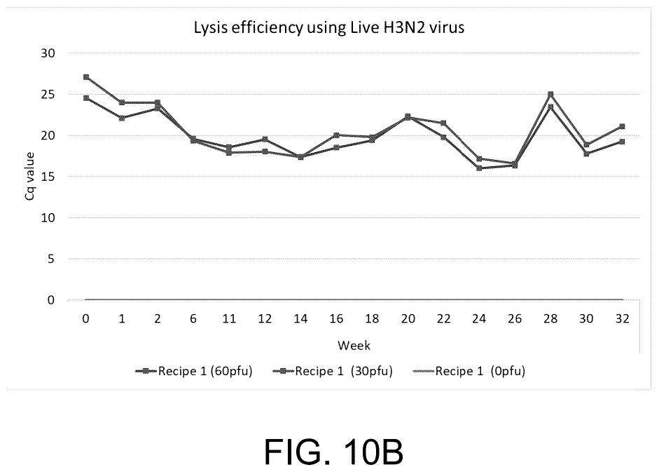

Example 10 illustrates the stability and lysis efficiency of the direct lysis buffer used with the lyophilized colorimetric LAMP reaction mixture on RNA or live virus. The intercalating fluorescence dye was added into the colorimetric LAMP reaction mixture to use fluorescence signal as indicator of performance. In this example, to ascertain the stability and performance of the direct lysis buffer, both the live virus FluA H3N2 and the RNA extracted from the virus FluA H3N2 were used for testing. The performance of the direct lysis buffer was challenged using 32×LoD (7.5TCID50/reaction) and 4×LoD (0.9375TCID50/reaction) of the RNA sample together with 8×LoD (60 pfu) and 4×LoD (30 pfu) of the live virus sample. FIG. 10A and FIG. 10B show real time stability and performance of the direct lysis buffer for the extracted RNA and the live virus, respectively. As shown in the figure, the performance for the 32nd week was comparable with the 6th week with ΔCq less than 2Cq for the extracted RNA, while for the live virus, ΔCq was less than 3Cq. Therefore, the performance of the direct lysis buffer was comparable with the freshly prepared buffer up to the 32nd week of preparation. The stability study was on-going until the 60th week and the result showed that the direct lysis buffer is able to be stored at room temperature to the 60th week without losing its performance.

From the above, the present disclosure provides the colorimetric LAMP system for detecting viral genomic materials with high sensitivity, high specificity, and reduced reaction time. Particularly, the present disclosure provides the recipe for colorimetric LAMP reaction mixture which allows for fast and efficient LAMP amplification and detection with color changes, and for reserving the functionality of the major components, especially the enzymes in the colorimetric LAMP reaction mixture during lyophilization process, and the direct lysis buffer system which is compatible to the colorimetric LAMP reaction mixture. The colorimetric LAMP system has the following advantages.

The direct lysis buffer is able to effectively lyse and release the nucleic acids from the viruses in nasal samples. Meanwhile, the buffer keeps the nucleic acids intact for downstream nucleic acid amplification. Normally, the lead time for the commercial spin column extraction method or magnetic beads based method may take about 30 minutes or more. In contrast, the lead time for the present direct lysis buffer can take less than 2 minutes, and preferably less than 1 minute, with comparable lysis efficiency on the target viruses. In addition, the present direct lysis buffer effectively removes the inhibitors in the sample without purification step, which is commonly used in other nucleic acid isolation procedures. This leads to a robust buffer system for better sensitivity.

In addition, the composition of the direct lysis buffer with optimal concentration is not only compatible with but also promotes the colorimetric LAMP reaction. Further, the direct lysis buffer is adjusted to the optimal starting pH and with proper buffering system to maintain the initial sample pH, meanwhile, to ensure the buffer is pH sensitive when the LAMP reaction starts in order to capture the pH signal shifting for the positive sample. Therefore, the direct lysis buffer is a pH sensitive and stable buffer system for signal differentiation.

Particularly, the colorimetric LAMP reaction mixture is able to be all-in-one lyophilized into the lyophilized bead or cake. The recipe of the colorimetric LAMP reaction mixture is specially formulated to include both enzymes and primers into single bead/cake to avoid different lyophilization process which was reported in the prior art. The selected lyoprotectant sugars allow the reagent to be lyophilized in reduced volume (less than 10 μl) which leads to much smaller bead/cake size. It contains morphologically stable and spherical shape with comparable performance with liquid reagent. Further, the lyophilized reagent can be rehydrated in seconds and does not generate bubbles after rehydration to avoid any disturbance during optical signal detection. The lyophilized reagent has excellent room temperature stability, and can be stored, transported and used at room temperature after rehydration with the direct lysis buffer.

Moreover, the colorimetric LAMP system has short turnaround time. The colorimetric LAMP reaction can be completed within 20 minutes, which is faster than the PCR detection system that takes around 1 hour for RNA detection. The colorimetric LAMP system has distinguishable color change for positive amplification and the signal can be detected either with naked eye or any other optical detection system.

While the disclosure has been described in terms of what is presently considered to be the most practical and preferred embodiments, it is to be understood that the disclosure needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A colorimetric loop-mediated isothermal amplification (LAMP) system, comprising:
a colorimetric LAMP reaction mixture, which is all-in-one lyophilized, comprising:
a primer set, a strand-displacing polymerase and deoxyribonucleoside triphosphates for amplifying a target sequence;
a pH indicating dye in a concentration ranged 0.08 to 0.3 mM; and
a lyoprotectant sugar in a concentration ranged 1 to 10% (w/v), wherein the lyoprotectant sugar is selected from the group consisting of trehalose, raffinose, dextran, mannitol and mixtures thereof; and
an extraction-free lysis buffer, comprising:
potassium chloride in a concentration ranged 10 to 50 mM;
ammonium sulfate in a concentration ranged 10 to 50 mM; and
a detergent in a concentration ranged 0.5 to 6% (w/v), wherein the detergent is selected from the group consisting of 2-ethylhexan-1-ol;2-methyloxirane;oxirane (CAS number: 64366-70-7), secondary alcohol ethoxylate (CAS number: 84133-50-6), 2-[4-(2,4,4-trimethylpentan-2-yl)phenoxy]ethanol (CAS number: 9002-93-1), 2-[4-(2,4,4-trimethylpentan-2-yl)phenoxy]ethan-1-ol (CAS number: 9002-93-1), and {2-[3,4-bis(2-hydroxyethoxy)oxolan-2-yl]-2-(2-hydroxyethoxy)ethoxy}ethyl dodecanoate (CAS number: 9005-64-5) and mixtures thereof;
wherein the lyophilized colorimetric LAMP reaction mixture is rehydrated with the extraction-free lysis buffer to be ready for nucleic acid amplification and detection.

2. The colorimetric LAMP system according to claim 1, wherein the colorimetric LAMP reaction mixture further comprises a reverse transcriptase for converting a viral genomic RNA to a complementary DNA.

3. The colorimetric LAMP system according to claim 1, wherein the pH indicating dye is phenol red or neutral red.

4. The colorimetric LAMP system according to claim 1, wherein a volume of the colorimetric LAMP reaction mixture is less than 10 μl.

5. The colorimetric LAMP system according to claim 1, wherein the colorimetric LAMP reaction mixture comprises 2 to 7.5% (w/v) of trehalose and 2 to 7.5% (w/v) of raffinose.

6. The colorimetric LAMP system according to claim 1, wherein the colorimetric LAMP reaction mixture comprises 2 to 7.5% (w/v) of trehalose, 2 to 7.5% (w/v) of raffinose, 1 to 2.5% (w/v) of dextran, and 1 to 5% (w/v) of mannitol.

7. The colorimetric LAMP system according to claim 1, wherein the colorimetric LAMP reaction mixture further comprises a magnesium salt in a concentration ranged 4 to 12 mM.

8. The method according to claim 1, wherein the detergent in the extraction-free lysis buffer is 0.5 to 6% (w/v) of 2-ethylhexan-1-ol;2-methyloxirane;oxirane (CAS number: 64366-70-7).

9. The method according to claim 1, wherein the detergent in the extraction-free lysis buffer is 0.5 to 6% (w/v) of secondary alcohol ethoxylate (CAS number: 84133-50-6).

10. The method according to claim 1, wherein the detergent in the extraction-free lysis buffer is 0.5 to 6% (w/v) of 2-[4-(2,4,4-trimethylpentan-2-yl)phenoxy]ethanol (CAS number: 9002-93-1).

11. The method according to claim 1, wherein the detergent in the extraction-free lysis buffer is 0.5 to 6% (w/v) of 2-[4-(2,4,4-trimethylpentan-2-yl)phenoxy]ethan-1-ol (CAS number: 9002-93-1).

12. The method according to claim 1, wherein the detergent in the extraction-free lysis buffer is 0.5 to 6% (w/v) of {2-[3,4-bis(2-hydroxyethoxy)oxolan-2-yl]-2-(2-hydroxyethoxy)ethoxy}ethyl dodecanoate (CAS number: 9005-64-5).
